(19) United States
(12) Patent Application Publication
(10) Pub. No.: US 2021/0133553 A1
(43) Pub. Date: May 6, 2021

(54) TRAINING A MODEL

(71) Applicant: KONINKLIJKE PHILIPS N.V., EINDHOVEN (NL)

(72) Inventors: TEUN VAN DEN HEUVEL, DEVENTER (NL); BINYAM GEBREKIDAN GEBRE, ROSMALEN (NL)

(21) Appl. No.: 16/640,101

(22) PCT Filed: Aug. 30, 2018
(86) PCT No.: PCT/EP2018/073286

§ 371 Date: Feb. 19, 2020

Related U.S. Application Data
(60) Provisional application No. 62/557,892, filed on Sep. 13, 2017.

(51) Int. Cl. G06N 3/08; G06F 40/169; G06N 3/04; G16H 30/40

(52) U.S. Cl. CPC G06N 3/08; G16H 30/40; G06N 3/04; G06F 40/169

(57) ABSTRACT

There is provided a computer-implemented method (200) and system for training a model. A first user input is received to annotate a first parameter in a portion of data (202). The first model is used to predict an annotation for at least one other parameter of the portion of data based on the received first user input for the first parameter (204). The annotated first parameter, the predicted annotation of the at least one other parameter and the portion of data are used as training data to train a second model (206).10

### TECHNICAL FIELD

Various embodiments described herein relate to the field of machine learning. More particularly, but not exclusively, various embodiments relate to a method and system for training a model.

### BACKGROUND

In many applications of machine learning, a set of annotated examples (e.g. training data) are provided to a machine learning procedure. The machine learning procedure uses the training data to develop a model that can be used to label new, previously unseen data. Manually annotating data for use as training data can be time consuming (costly) and boring for the annotator (possibly affecting quality), particularly where large sets of training data comprising hundreds or thousands of annotated examples are needed. Moreover, it is often not known beforehand which parameters will be most important to the machine learning model, or how many different annotations will be needed to train the model. This can lead to unnecessary or redundant annotation by the user, which is frustrating for the annotator and wasteful.

In particular, annotation of a single data sample may consist of multiple annotator actions, some of which may be redundant in hindsight. For example, precisely annotating the location of a stent in interventional x-ray (iXR) data generally requires the annotator to input two clicks per frame (corresponding to the two ends of the stent in each image), while such precision may not be required for the complete dataset only for a limited subset.

### SUMMARY

As noted above, a limitation with existing approaches is that they tend to incorporate burdensome and potentially unnecessary manual annotation of training datasets. This can be inconvenient, for example, if more than one parameter is required per example or if the annotation needs to be performed by a busy trained professional such as a medical professional (whose time may be expensive).

One known approach to address this problem is to intermittently annotate data and train (e.g. update) the model, for example, by providing the machine learning procedure with correctly annotated examples of data for which the model has previously made incorrect predictions. While this can reduce the annotation burden, regularly updating a model may be inconvenient. Furthermore, this type of training may still involve the expenditure of potentially unnecessary effort, particularly where the person annotating the data annotates more than one parameter per sample.

Another known approach to avoid bulk annotations is reinforcement learning, whereby model predictions are rated (by a human) as correct or incorrect and this coarse feedback is used to improve the model. With reinforcement learning, although annotation/feedback effort is reduced, the notion of precise annotations may also be completely abandoned, which may not be optimal for performance of the resulting model. An example of this type of learning method is provided in US 2010/0306141.

There is therefore a need for a more efficient method and system for training a model that overcomes some of the aforementioned issues.

Therefore, according to a first aspect, there is provided a computer-implemented method of training a model. The method includes receiving a first user input to annotate a first parameter in a portion of data, using a first model to predict an annotation for at least one other parameter of the portion of data based on the received first user input for the first parameter, and using the annotated first parameter, the predicted annotation of the at least one other parameter and the portion of data as training data to train a second model.

In some embodiments, the second model may be for annotating the first parameter and the at least one other parameter in a further portion of data.

In some embodiments, the method may further include forming a training set of training data for training the second model by repeating, for a plurality of portions of data, receiving a first user input and using a first model to predict an annotation.

In some embodiments, using a first model to predict an annotation may be further based on the portion of data.

In some embodiments, the method may further include receiving a second user input providing an indication of an accuracy of the predicted annotation of the at least one other parameter and using the indication of the accuracy of the predicted annotation as training data to train the second model.

In some embodiments, the method may further include updating the first model based on the received second user input and the predicted annotation of the at least one other parameter.

In some embodiments, using a first model to predict an annotation may include using the first model to provide a plurality of suggestions for the annotation of the at least one other parameter and the method may further include receiving a third user input indicating an accuracy of at least one of the plurality of suggestions and using the indicated accuracy of the at least one of the plurality of suggestions as training data to train the second model.

In some embodiments, the method may further include updating the first model based on the received third user input and the plurality of suggestions.

In some embodiments, the predicted annotation of the at least one other parameter may be based on confidence levels calculated by the first model.

In some embodiments, the portion of data may include an image, the first parameter may represent a location of a first feature in the image, and the at least one other parameter may represent locations of one or more other features in the image.

In some embodiments, the portion of data may include a sequence of images separated in time, the first parameter may relate to a first image in the sequence of images, and the first model may predict an annotation of the first parameter and/or the at least one other parameter of the portion of data in a second image in the sequence of images. In these embodiments, the second image may be a different image to the first image.

In some embodiments, the portion of data may include medical data.

In some embodiments, the first and/or second model may include a deep neural network.

According to a second aspect, there is provided a non-transitory computer readable medium, the computer readable medium having computer readable code embodied therein, the computer readable code being configured such that, on execution by a suitable computer or processor, the computer or processor is caused to perform the method described above.

According to a third aspect, there is provided a system including a memory including instruction data representing a set of instructions and a processor configured to communicate with the memory and to execute the set of instructions. The set of instructions, when executed by the processor, cause the processor to receive a first user input to annotate a first parameter in a portion of data, use a first model to predict an annotation for at least one other parameter of the portion of data based on the received first user input for the first parameter, and use the annotated first parameter, the predicted annotation of the at least one other parameter and the portion of data as training data to train a second model.

According to the aspects and embodiments described above, the limitations of existing techniques are addressed. In particular, according to the above-described aspects and embodiments, a difficult task can be split into two simpler ones that overall require less user annotation effort. By using a first model to predict annotations for use in training a second model in this way, the number of annotations that are required from the user is reduced. This saves the user time, particularly when annotating multi-parameter data and makes the training process overall more efficient. Moreover, in view of the fact that the first model is used to predict the annotation for the at least one other parameter based on the first user input, as opposed to independently annotating both the first parameter and the at least one other parameter straight away, the initial amount of fully annotated training data needed to train the first model (and thus the annotation burden on the user) is significantly reduced.

There is thus provided a more efficient method and system for training a model, which overcomes the existing problems.

### BRIEF DESCRIPTION OF THE DRAWINGS

For a better understanding of the embodiments, and to show more clearly how they may be carried into effect, reference will now be made, by way of example only, to the accompanying drawings, in which:

FIG. 1 is a block diagram of a system according to an example embodiment;

FIG. 2 illustrates an example computer-implemented method according to an embodiment;

FIG. 3 illustrates an example process according to an embodiment;

FIG. 4 illustrates a further example of a process according to an embodiment;

FIG. 5 illustrates a block diagram of an example system architecture according to an embodiment;

FIG. 6 illustrates a standard annotation method for locating the ends of a stent in a medical image;

FIG. 7 illustrates a manner in which embodiments of the method and system described herein may be applied to locate the ends of a stent in a medical image; and

FIG. 8 is a schematic diagram of an example first model according to an embodiment.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

As noted above, there is provided an improved method and system for training a model, which overcomes some of the existing problems.

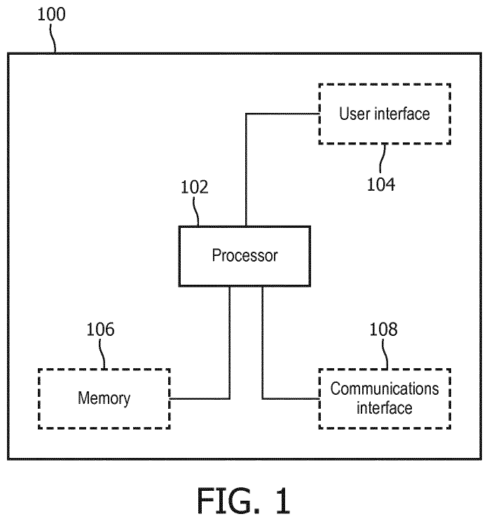

FIG. 1 shows a block diagram of a system 100 according to an embodiment that can be used for training a model. With reference to FIG. 1, the system 100 comprises a processor 102 that controls the operation of the system 100 and that can implement the method described herein. The system 100 may further include a memory 106 including instruction data representing a set of instructions. The memory 106 may be configured to store the instruction data in the form of program code that can be executed by the processor 102 to perform the method described herein. In some implementations, the instruction data can include a plurality of software and/or hardware modules that are each configured to perform, or are for performing, individual or multiple steps of the method described herein. In some embodiments, the memory 106 may be part of a device that also includes one or more other components of the system 100 (for example, the processor 102 and/or one or more other components of the system 100). In alternative embodiments, the memory 106 may be part of a separate device to the other components of the system 100.

The processor 102 of the system 100 can be configured to communicate with the memory 106 to execute the set of instructions. The set of instructions, when executed by the processor may cause the processor to perform the method described herein. The processor 102 can include one or more processors, processing units, multi-core processors or modules that are configured or programmed to control the system 100 in the manner described herein. In some implementations, for example, the processor 102 may include a plurality of processors, processing units, multi-core processors and/or modules configured for distributed processing. It will be appreciated by a person skilled in the art that such processors, processing units, multi-core processors and/or modules may be located in different locations and may each perform different steps and/or different parts of a single step of the method described herein.

Briefly, the set of instructions, when executed by the processor 102 of the system 100 cause the processor 102 to receive a first user input to annotate a first parameter in a portion of data, use a first model to predict an annotation for at least one other parameter of the portion of data based on the received first user input for the first parameter and use the annotated first parameter, the predicted annotation of the at least one other parameter and the portion of data as training data to train a second model.

The technical effect of the system 100 may be considered to be splitting a difficult task into two simpler ones that overall require less user annotation. By using a first model to predict annotations for training data for use in training a second model in this way, the number of annotations that are required from the user is reduced. This saves the user time, particularly when annotating multi-parameter data and makes the training process overall more efficient.

In some embodiments, the set of instructions, when executed by the processor 102 may also cause the processor 102 to control the memory 106 to store data and information relating to the methods described herein. For example, the memory 106 may be used to store any of the portion of data, the first user input, the first parameter, the first model, the predicted annotation for the at least one other parameter of the portion of data, the second model, or any other data or information, or any combinations of data and information, which results from the method described herein.

In any of the embodiments described herein, the portion of data may include any data that can be processed by a model (such as a machine learning model). For example, the portion of data may include any one or any combination of: text, image data, sensor data, instrument logs and/or records. In some embodiments, the portion of data may include medical data such as any one or any combination of medical images (for example, images acquired from a CT scan, X-ray scan, or any other suitable medical imaging method), an output from a medical instrument or sensor (such as a heart rate monitor, blood pressure monitor, or other monitor) or medical records. Although examples have been provided of different types of portions of data, a person skilled in the art will appreciate that the teachings provided herein may equally be applied to any other type of data that can be processed by a model (such as a machine learning model).

As mentioned earlier, the processor 102 of the system 100 is caused to receive a first user input to annotate a first parameter in the portion of data. In some embodiments, as illustrated in FIG. 1, the system 100 may include at least one user interface 104 configured to receive the first user input (and/or any of the other user inputs described herein). The user interface 104 may allow a user of the system 100 to manually enter instructions, data, or information to annotate the first parameter in the portion of data. The user interface 104 may be any type of user interface that enables a user of the system 100 to provide a user input, interact with and/or control the system 100. For example, the user interface 104 may include one or more switches, one or more buttons, a keypad, a keyboard, a mouse, a touch screen or an application (for example, on a tablet or smartphone), or any other user interface, or combination of user interfaces that enables the user to indicate a manner in which the first parameter is to be annotated in the portion of data.

In some embodiments, the user interface 104 (or another user interface of the system 100) may enable rendering (or output or display) of information, data or signals to a user of the system 100. As such, a user interface 104 may be for use in providing a user of the system 100 (for example, a medical personnel, a healthcare provider, a healthcare specialist, a care giver, a subject, or any other user) with information relating to or resulting from the method according to embodiments herein. The processor 102 may be configured to control one or more user interfaces 104 to provide information resulting from the method according to embodiments herein. For example, the processor 102 may be configured to control one or more user interfaces 104 to render (or output or display) the portion of data, the first user input, the first parameter, the annotation from the first input, the predicted annotation for the at least one other parameter, information pertaining to the first and/or second models, or any other information, or any combination information, which results from the method described herein. For example, the user interface 104 may include a display screen, a graphical user interface (GUI) or other visual rendering component, one or more speakers, one or more microphones or any other audio component, one or more lights, a component for providing tactile feedback (e.g. a vibration function), or any other user interface, or combination of user interfaces for providing information relating to, or resulting from the method, to the user. In some embodiments, the user interface 106 may be part of a device that also includes one or more other components of the system 100 (for example, the processor 102, the memory 104 and/or one or more other components of the system 100). In alternative embodiments, the user interface 106 may be part of a separate device to the other components of the system 100.

In some embodiments, as illustrated in FIG. 1, the system 100 may also include a communications interface (or circuitry) 108 for enabling the system 100 to communicate with any interfaces, memories and devices that are internal or external to the system 100. The communications interface 108 may communicate with any interfaces, memories and devices wirelessly or via a wired connection.

It will be appreciated that FIG. 1 only shows the components required to illustrate this aspect of the disclosure and, in a practical implementation, the system 100 may include additional components to those shown. For example, the system 100 may include a battery or other power supply for powering the system 100 or means for connecting the system 100 to a mains power supply.

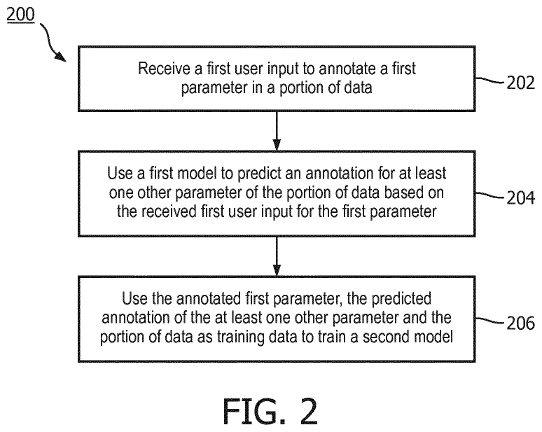

FIG. 2 illustrates a computer-implemented method 200 of training a model according to an embodiment. The illustrated method 200 can generally be performed by or under the control of the processor 102 of the system 100. The method may be partially or fully automated according to some embodiments.

Briefly, with reference to FIG. 2, the method includes receiving a first user input to annotate a first parameter in a portion of data (at block 202 of FIG. 2) and using a first model to predict an annotation for at least one other parameter of the portion of data based on the received first user input for the first parameter (at block 204 of FIG. 2). The method also includes using the annotated first parameter, the predicted annotation of the at least one other parameter and the portion of data as training data to train a second model (at block 206 of FIG. 2).

In more detail, at block 202 of FIG. 2, a user input is received to annotate a first parameter in a portion of data. As described in detail earlier with respect to system 100 of FIG. 1, the portion of data may include any data that can be processed by a model (such as a machine learning model), such as textual or image data, including, but not limited to medical data, such as medical images, instrument data (such as sensor data) and/or medical records.

Generally, the first parameter includes any information about the portion of data that can be supplied to a model to aid the model process the contents of the portion of data to produce a desired output. For example, the first parameter may include one or more numbers associated with the portion of data, one or more classes associated with the portion of data or one or more alphanumeric strings associated with the portion of data. The first parameter may be associated with a feature of the portion of data. Examples of features include, but are not limited to, measurable properties, observed properties, derived properties or any other properties (or characteristics) of the portion of data, or any combinations of properties (or characteristics) of the portion of data. In some embodiments, the first parameter may relate to the user's interpretation of an aspect of the first portion of data. For example, the first parameter may relate to a user classification of the portion of data (e.g. the user may indicate the observed content in the portion of data, or the manner in which to classify one or more aspects of the portion of data).

In some embodiments, the first parameter may include information derived by the user from the portion of data (for example, where the portion of data includes medical data, the first parameter may include a diagnosis, based on the user's interpretation of the first portion of data). The first parameter may include the location of a feature in the portion of data. For example, in embodiments where the portion of data is an image, the first parameter may include the location of a feature in the image. Where the image is a medical image, the feature may include the location of an anatomical structure, an artificial structure and/or an abnormality (such as diseased or damaged tissue) in the medical image. More generally, the first parameter may include the user's interpretation of the content shown in the image (for example, the user may indicate that the image relates to a “heart”).

The user can annotate the first parameter in the portion of data by providing an indication of the annotation (which may be, for example, a number, classification, set of co-ordinates or text) to associate with the parameter. The received first user input to annotate the first parameter may take any form. For example, where the parameter includes the location of a feature in a portion of image data, the portion of image data may be rendered on a user interface 104 and the user may indicate the position of the feature in the image using a user interface 104, such as a mouse, touch screen, or any other type of user interface suitable for indicating the position of the feature in the image. The first user input may therefore include a mouse click or a touch on a screen indicating the position of the feature in the image. In other embodiments, the first user input may include text input, for example, by means of a keyboard. The skilled person will appreciate that these are merely exemplary however and that there are many other methods of providing a first user input to annotate a first parameter in the portion of data.

Returning back to FIG. 2, at block 204, the method includes using a first model to predict an annotation for at least one other parameter of the portion of data, based on the received first user input for the first parameter.

Generally, the first model includes any model that uses the annotated first parameter (as derived from the first user input) to predict an annotation for at least one other parameter of the portion of data. In some embodiments, a model can be a model that outputs completion suggestion(s) based on a partial annotation and the corresponding input data. Functionally, the first model may be an autocomplete model or autocomplete algorithm, whereby the model predicts, based on the user input to annotate the first parameter, the manner in which the user may annotate at least one other parameter. In other words, the first model may auto-complete or predict future user behaviour (i.e. future annotations) from previous user actions (i.e. previous user annotation(s)).

In some embodiments, the first model may be a hard-coded model. A hard-coded model may, for example, process the annotation of the first parameter according to a set of coded rules or criteria in order to predict an annotation for the at least one other parameter. The coded rules may, for example, be based on spatial and/or temporal patterns observed by a user in annotations of other examples of portions of data.

In alternative embodiments, the first model may be a machine learning model. The first model, for example, may be a deep learning machine learning model. In some embodiments, the first model may include a deep neural network. The skilled person will appreciate however that the first model can be any other sort of model that can be used to predict an annotation for at least one other parameter, based on a received first user input to annotate a first parameter. In some embodiments, the first model may predict the annotation for the at least one other parameter in the portion of data, based on annotations that were provided for previous examples of portions of data (e.g. based on annotated training data). The first model may predict the manner in which the user may annotate the at least one other parameter, based on the first user input and optionally also based on patterns observed in the manner in which the user previously annotated the first and/or the at least one other parameter in the training data.

In some embodiments, the predicted annotation for the at least one other parameter may be based on confidence levels (or limits) calculated by the first model. For example, a prediction may be made if the model has a confidence in the prediction that is above a predetermined threshold. The skilled person will appreciate that an appropriate threshold value for the predetermined threshold may depend on the particular goals and implementation of the system. However, as examples, a prediction may be chosen if the model has more than (or more than about) fifty percent confidence that the prediction is correct, more than (or more than about) sixty percent confidence that the prediction is correct, more than (or more than about) seventy percent confidence that the prediction is correct or more than (or more than about) eighty percent confidence that the prediction is correct.

The person skilled in the art will be familiar with methods suitable for training a model (such as a machine learning model). For example, the first model may initially be trained using fully annotated portions of data. For example, the user may initially annotate the first parameter and the at least one other parameter for an initial batch of portions of data. This initial batch of fully annotated data may be used to train the first model to predict an annotation for the at least one other parameter from a user annotated first parameter.

In some embodiments, the first model may be improved using user feedback. For example, the method may include receiving a second user input providing an indication of an accuracy of the predicted annotation of the at least one other parameter. For example, the user may indicate whether the prediction is correct or incorrect. If the prediction is incorrect, the user may provide a correct annotation for the at least one other parameter.

The method may, in some embodiments, further include updating the first model based on the received second user input and the predicted annotation of the at least one other parameter. In embodiments where the user provides a corrected annotation, the first model may, for example, be updated using the correct annotation as further training data. In embodiments where the user provides as a second input a confirmation that the predicted annotation is correct, the first model may, for example, be updated using the confirmed predicted annotation as further training data.

In view of the fact that the first model is trained to predict the annotation for the at least one other parameter from a first user input, as opposed to independently annotating both the first parameter and the at least one other parameter straight away, the initial amount of fully annotated training data needed to train the first model (and thus the annotation burden on the user) is significantly reduced.

In some embodiments, at block 204 of FIG. 2, the first model may provide a plurality of suggestions of the manner in which the at least one parameter may be annotated. For example, the model may determine that there are different possible annotations for a particular other parameter, in view of the received first user input. The different possible annotations may be presented to the user as suggestions for the manner in which the parameter may be annotated. In some embodiments, the suggestions may result from confidence levels (or limits) calculated by the first model. For example, the first model may determine two (or more) annotations for a parameter, in view of calculated confidence levels. The first model may include the two or more annotations in the plurality of suggestions, or alternatively select a subset to present to the user. For example, the plurality of suggestions may include all possible annotations having a confidence (as determined by the first model) above a predetermined threshold. As will be appreciated by the person skilled in the art, the appropriate level for the predetermined threshold may depend on the application and the desired accuracy (an appropriate predetermined threshold level may therefore, for example, be determined through experimentation, or be configurable by the user). In some applications, the method may include presenting all options for the annotation of a parameter to the user for which the first model has a confidence higher than, for example, 20 percent (or about 20 percent) or 30 percent (or about 30 percent) confidence. The person skilled in the art will appreciate however that these values are merely exemplary and that the predetermined threshold may be set at any appropriate level.

In some embodiments, the plurality of suggestions may be provided to the user (for example, using a user interface 104). The method may further include receiving a third user input, indicating an accuracy of at least one of the plurality of suggestions. For example, the third user input may rank the suggestions in order of accuracy, or provide an indication of which suggestion is the optimal suggestion of the plurality of suggestions. If the user considers the suggestions to be incorrect, then the third user input may indicate than none of the suggestions are correct and/or provide a corrected annotation for the parameter. The first model may then be updated, based on the third user input (e.g. the indicated accuracy the plurality of suggestions) and the plurality of suggestions provided by the first model. In embodiments where the user provides a corrected annotation, the first model may, for example, be updated using the corrected annotation as further training data.

In some embodiments, at block 204 of FIG. 2, using a first model to predict an annotation may be further based on the portion of data. For example, the first model may analyse the portion of data to determine one or more features (such as those described above with respect to block 202) associated with the portion of data that can be used to predict the annotation of the at least one other parameter. In this way, the method may further include data analysis steps, such as text recognition, language processing and/or image analysis on the portion of data to derive the one or more features that can be used in addition to the first user input to annotate the first parameter to predict the annotation of the at least one other parameter.

In some embodiments, the annotator may be prompted to indicate any further required parameters that have not yet been annotated (either by the user or the first model) and/or confirm completeness of the parameters.

It will be appreciated that, in some embodiments, the method described earlier with respect to blocks 202 (receiving a first user input) and 204 (using a first model to predict an annotation) of FIG. 2 may be repeated for a plurality of different portions of data, so as to form a training set of training data (e.g. annotated portions of data) for use in training the second model.

Turning now to block 206 of FIG. 2, the method includes using the annotated first parameter (which is received from the user at block 202 of FIG. 2), the predicted annotation for the at least one other parameter (as predicted by the first model at block 204 of FIG. 2) and the portion of data, as training data to train a second model. In this way, the output of the first model is used to train the second model. The fact that annotations predicted by the first model are used to train the second model reduces the annotation burden on the user because the first model is used to provide some of the annotations that would otherwise have to be provided by the user.

In some embodiments, the second model may be a machine learning model. The second model may be, for example, a deep learning machine learning model. In some embodiments, the second model may include a deep neural network. Although examples have been provided for the type of model that can be used for the second model, the person skilled in the art will appreciate that the methods herein apply equally to any other models that are trained using annotated training data. In some embodiments, the second model may be for annotating the first parameter and/or the at least one other parameter in one or more further (e.g. unseen) portions of data. In effect, the second model may be for independently predicting annotations of the first and the at least one other parameter in other portions of data, without user input, e.g. without relying directly on any (partial) annotation.

In some embodiments, outputs of the training process of the first model can be input to the training procedure as additional training data for the second model. For example, it was described above with respect to block 204 of FIG. 2 that, in some embodiments when training the first model, the method includes receiving a second user input providing an indication of an accuracy of the at least one other parameter. In this sense, the user may provide feedback on the predicted annotation of the at least one other parameter. In some embodiments, alternatively or additionally, the indication of the accuracy of the predicted annotation made by the first model may be used as training data to train the second model. For example, if the user indicates that an annotation of a parameter predicted by the first model is a “high quality” parameter, then the second model may place a higher weighting to this annotation in the training procedure of the second model than to other annotations that the user has indicated as being “medium” or “poor” quality annotations. In some embodiments, if the user indicates that an annotation of a parameter predicted by the first model is incorrect, then the second model may further learn from the incorrect annotation (e.g. the training procedure for the second model may learn from the first model's mistakes).

More generally, the annotation for the first parameter and/or the annotation for the at least one other parameter may be rated for reliability according to whether the annotation was made by the user or by the first model. Annotations may be rated as more reliable if they are made by the user as opposed to the first model. In some embodiments, each annotation (e.g. the annotation for the first parameter and/or the annotation for the at least one other parameter) may be rated for reliability on a scale of “user annotated”, “model annotated and checked by user” or “model annotated”. These ratings can then be used as training data with which to train the second model. For example, the second model may take the ratings into account when learning from the training data, by giving most weight to “user annotated” parameters, less weight to “model annotated and checked by user” parameters and least weight to “model annotated” parameters, as noted above.

As was also described above with respect to block 204 of FIG. 2, in some embodiments, the first model can be used to provide a plurality of suggestions for the annotation of the at least one parameter, and the method may include receiving a third user input indicating an accuracy of at least one of the plurality of suggestions. The indication of an accuracy of at least one of the plurality of suggestions may also be used as training data for the second model. Thus, in some embodiments, the method may further include using the indicated accuracy of the at least one of the plurality of suggestions as training data to train the second model. For example, the second model may take the indicated accuracy of the at least one of the plurality of suggestions into account when learning from the training data, by giving most weight to the most accurate suggestions of the plurality of suggestions. In this way, the second model may learn not only from the successful annotations made by the first model, but also from the less successful or even unsuccessful predicted annotations made by the first model.

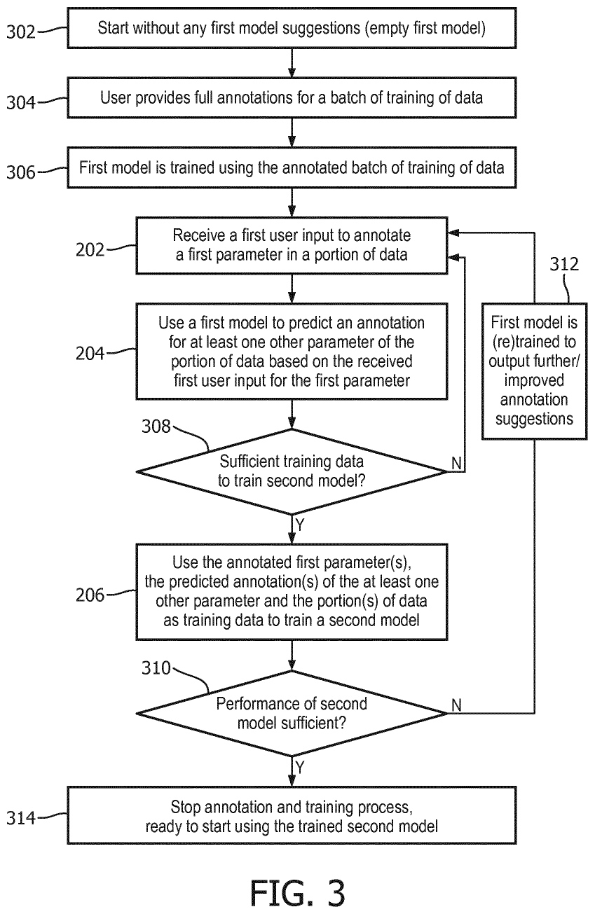

FIG. 3 is a process diagram further illustrating some of the preceding ideas. In block 302 of FIG. 3, the process starts with an empty first model. For example, the process starts without any first model suggestions. In block 304 of FIG. 3, the user provides full annotations for an initial (new) batch of training data. Each user annotated portion of data in the initial batch of training data is used to train the first model in block 306 of FIG. 3. Examples of the manner in which to train the first model were described earlier where the first model was introduced, in the section relating to block 204 of FIG. 2 and these examples will be understood to apply to block 306 of FIG. 3.

Once the first model is trained, it can be used to annotate further portions of data according to the method 200 described above with respect to FIG. 2. Thus, at block 202 of FIG. 3, the process includes receiving a first user input to annotate a first parameter in a portion of data (as described earlier with respect to block 202 of FIG. 2). At block 204 of FIG. 3, the process may include using a first model to predict an annotation for at least one other parameter of the portion of data based on the received first user input for the first parameter (as described earlier with respect to block 204 of FIG. 2). In this way, the portion of data is annotated (by the user and the first model) for use in training the second model. In some embodiments, at block 308 of FIG. 3, the method includes determining whether sufficient annotated portions of data are available to train the second model.

In some embodiments, the step of determining whether sufficient annotated portions of data are available may include comparing the number of annotated portions of data to a predetermined threshold. In some examples, the predetermined threshold may be set based on numerical analysis (e.g. simulations relating to the performance of the second model for different sizes of training data). In other examples, the threshold may be set by the user based on, for example, previous experience. In some embodiments, block 308 of FIG. 3 can be regarded as a batching mechanism, whereby the user can periodically review whether sufficient training data is available, before the second model is (re)trained. This can be more efficient compared to (re)training the second model after each new annotation, if the user has to wait between annotations whilst the model is updated. In this way, the user's time can be utilised more effectively.

If insufficient portions of annotated data are available, then blocks 202 and 204 of FIG. 3 may be repeated on further portions of data until it is determined that enough annotated portions of data are available to train the second model. At block 206 of FIG. 3, the annotated first parameter(s), the predicted annotation(s) of the at least one other parameter and the portion(s) of data are used as training data to train the second model. The second model may be trained using any of the techniques outlined earlier with respect to block 206 of FIG. 2.

After this training, the performance of the second model is reviewed at block 310 of FIG. 3 to check whether the performance of the second model is sufficient. If the performance of the second model is insufficient (e.g. not accurate enough for the user's purpose), the process moves to block 312 of FIG. 3 whereby the first model is retrained to output further, improved annotation suggestions of further portions of data. Block 312 may include, for example, re-training the first model based on a user input indicating the accuracy of predicted annotations of the first model (as was described above with respect to block 204 of FIG. 2). In some embodiments, at block 312, the user may provide further batch(es) of fully annotated training data with which to train the first model. By reviewing the performance of the second model in this way, an experimental approach is effectively enabled, such that the model is updated in an iterative fashion. This may be more efficient than generating (potentially unnecessarily) large sets of training data from the outset. As part of the process of retraining the second model, blocks 202, 204, 308, 206, 310 and 312 of FIG. 3 may be repeated until the performance of the second model is sufficient. When the performance of the second model is sufficient, the process moves to block 314 of FIG. 3 where the annotation and training process is stopped and the trained second model is ready to use.

In this way, a parallel learning track is used to provide suggestions for partial annotations. This is based on the insight that training a model to complete partial annotations is easier (i.e. requires less fully annotated data to reach some performance level) than the training of the eventual full model. Moreover, even if the first model has a suboptimal performance level, it can still be useful in the sense of improving the annotation process efficiency.

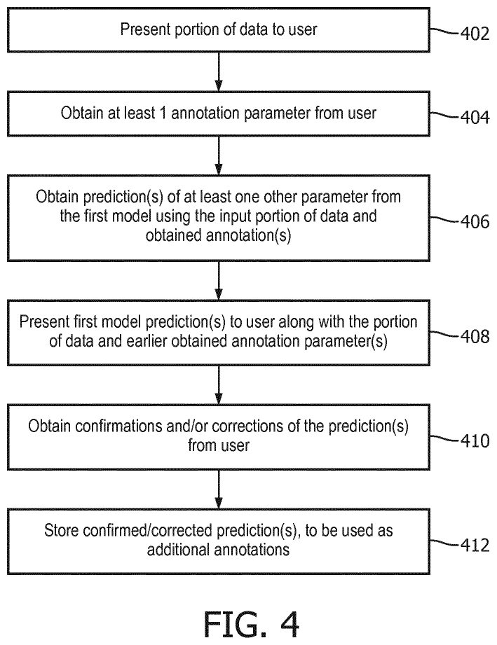

FIG. 4 illustrates a process for producing annotated training data using the first model. The process in FIG. 4 can generally be used in block 306 of FIG. 3. In block 402 of FIG. 4, a first portion of data is presented to the user for the user to annotate, for example, using a user interface 104 as described earlier. At block 404, one or more annotations are received (or obtained) from the user according to any of the methods outlined earlier with respect to block 202 of FIG. 2. One or more predictions of an annotation for at least one other parameter are then obtained from the first model in block 406 of FIG. 2 (according to any of the methods described earlier with respect to block 204). In block 408 of FIG. 4, the predictions of the first model are presented to the user, along with the first portion of data and the user annotated parameters obtained at block 404 of FIG. 4. At block 410 of FIG. 4, confirmations and/or corrections of the predictions are obtained from the user and, at block 412 of FIG. 4, the annotations (as confirmed or corrected by the user) are stored for use as additional annotations. The additional annotations can then be used as training data to train the second model in the manner described earlier with reference to block 206 of FIG. 2 or FIG. 3.

In this way, annotations produced by the first model can be stored for use in training the second model, including any information or feedback received by the user.

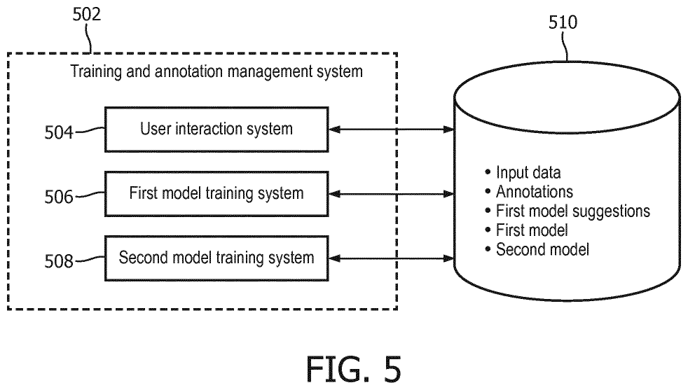

FIG. 5 shows an example architecture of a training and annotation management system 502 that can form part of the system 100 of FIG. 1 for implementing the processes illustrated in FIGS. 3 and 4. The training and annotation management system 502 may include a subsystem 504 related to interacting with the user. For example, the subsystem 504 may instruct the processor 102 of the system 100 to receive the first user input to annotate the first parameter in the portion of data. The subsystem 504 may further instruct the processor 102 of the system 100 to render the portion of data, or any other data or information on a user interface 104. The training and annotation management system 502 may further include a subsystem 506 relating to training the first model. For example, the subsystem 506 may instruct the processor 102 of the system 100 to implement any of the processes for training the first model described earlier, for example, with respect to block 204 of FIG. 2. The training and annotation management system 502 may further include a subsystem 508 for training the second model. For example, the subsystem 508 may instruct the processor 102 of the system 100 to implement any of the training processes for training the second model as described earlier with respect to block 206 of FIG. 2.

Generally, the training and annotation management system 502, may interact with one or more databases 510 included in one or more memories (such as the memory 106 of the system 100). Such databases may be used, for example, to store any data input by the user, any annotations (either provided by the user, or predicted by the first model), the first model, the second model and/or any other information provided or associated with any of the methods and processes described herein.

Turning now to another example, in some embodiments, the portion of data may include an image, the first parameter may represent a location of a first feature in the image, and the at least one other parameter may represent locations of one or more other features in the image. For example, the user may provide a user input indicating the location of the left hand in the image, from which the first model may predict the location of the right hand. The first model may determine the locations of the one or more other features in the image through spatial patterns observed in previously annotated images (for example, spatial patterns observed between the location of the first feature in the image and the location of the one or more other features in the image in training data used to train the first model).

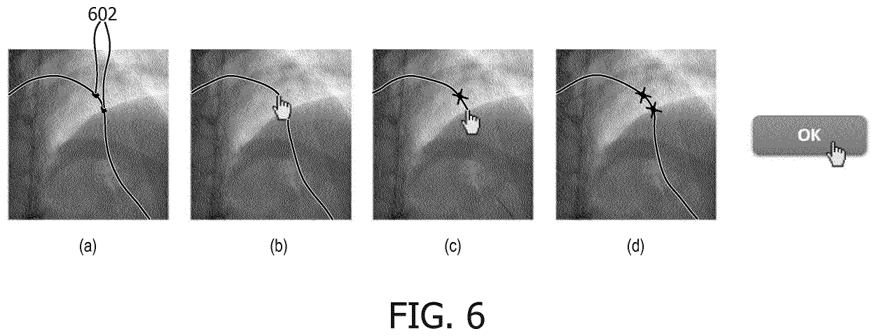

These embodiments are explained in more detail with respect to FIGS. 6, 7 and 8.

FIG. 6 illustrates an example of the manner in which labelling can be applied to medical image data according to a standard method. In this example, the second model is for use in localization of balloon marker pairs in interventional X-ray images, where the markers indicate the ends of a stent. In this case, the traditional annotation process consists of providing two coordinate pairs (e.g. x-y coordinates, possibly derived from mouse clicks on the image) for each image. FIG. 6a shows an example image showing a pair of balloon markers 602, FIG. 6b shows the user annotating the position of a first end of the stent in the image, and FIG. 6c shows the user annotating the position of the second end of the stent in the image. FIG. 6d illustrates the final annotated data, each annotated end of the stent being represented by a cross. At the end of the annotation process, the user may indicate that they are happy with the annotation, for example, by clicking on an “ok” button.

According to the embodiments herein, the traditional annotation process described above with respect to FIG. 6 is improved by training the first model to output the location of the second end of the stent, based on the input image and the user-input location of the first end of the stent. In this way, the number of annotations required by the user is halved, saving time and improving efficiency of the annotation.

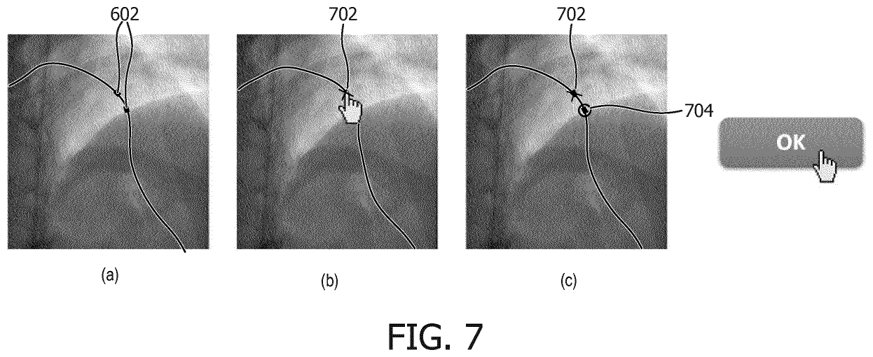

FIG. 7 illustrates the annotation process according to the embodiments described herein and shows the improvement over the standard annotation process illustrated in FIG. 6. In this example, the portion of data is an image of a stent, as shown in FIG. 7a. FIG. 7a shows the same pair of balloon markers 602 as in FIG. 6a. A first user input is received (for example, in the form of a mouse click), as shown in FIG. 7b. In this example, the first parameter is the location of a first balloon marker in the image, and the first user input annotates the location of the first balloon marker in the image, as indicated by the cross 702. Based on the received user input annotating the first parameter in the image, the first model is used to predict at least one other parameter, in this example, the location of the second balloon marker 704, as illustrated by the lower circle shown in FIG. 7c.

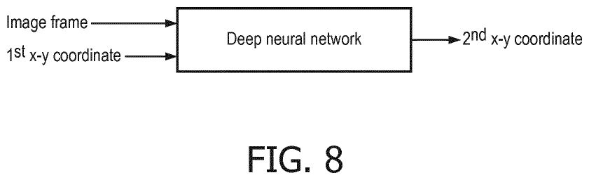

FIG. 8 illustrates an example of a suitable model for the first model that can be used in the example illustrated in FIG. 7. The suitable model here is a deep neural network. Essentially, the deep neural network takes as inputs the image of the stent (as shown in FIG. 7a) and an x-y co-ordinate of a first end of the stent as received in the first user input (as shown in FIG. 7b). The deep neural network outputs the location of the second end of the stent (as shown by the lower circle 704 in FIG. 7c). Details of how to design and train such a model are provided with respect to blocks 204 and FIG. 4 above and will be familiar to those skilled in the art of deep learning.

In this way, the number of annotations required from the user are reduced compared to the traditional annotation process illustrated in FIG. 6. This saves time for the user and has efficiency savings, which are beneficial in the medical field where the annotation process is likely to require the skills of a highly skilled (and therefore expensive) medical processional.

The examples above may be extended, such that, for example, the first model can be used to efficiently annotate a sequence of images, such as a sequence of images separated in time (e.g. a time sequence of images). In such examples, the user may annotate a first parameter that relates to a first image in the sequence of images and the first model may predict an annotation of the first parameter and/or the at least one other parameter of the portion of data in a second image in the sequence of images. The second image may be a different image to the first image. Generally, the parameter annotated in the different images in the sequence may be the same parameter as was annotated by the user in the first image (for example, if the user provides user input to annotate a left hand in one of the images of the sequence of images, the first model may annotate the location of the same left hand in one or more other images of the sequence). Alternatively, the first model may annotate a different parameter in the other image (for example, if the user provides user input to annotate a left hand in one of the images of the sequence of images, the first model may annotate the location of the right hand in one or more other images of the sequence).

In such embodiments including sequences of images, the model may predict an annotation of the at least one other parameter based on spatial and temporal patterns observed in previously annotated images (i.e. training data). In the example of stent localisation above, the predictions for nearby images in the sequence may therefore be based on temporal consistency of the manner in which the stent markers move over time. Combining spatial and temporal patterns makes the model suggestions more robust and efficient as the locations of parameters in image becomes predictable in the subsequent images in the sequence (for example, the stent markers remain a fixed separation over time and this can be tracked by the model). In this way, the method is able to offer fast and reliable annotation of sequences of images.

The embodiments described above provide advantages in that the first model is used to partially annotate training data for the second model, thus reducing the annotation burden on the user. Although the user may still need to provide some annotations in order to train the first model, far fewer annotations are generally required to train the first model than the second model and so the annotation burden on the user is decreased. Effectively, the embodiments herein break a large learning problem into two smaller ones, which require overall less training input from the user. In this way, the current method overcomes some of the problems associated with existing techniques.

There is also provided a computer program product including a computer readable medium, the computer readable medium having computer readable code embodied therein, the computer readable code being configured such that, on execution by a suitable computer or processor, the computer or processor is caused to perform the method or methods described herein. Thus, it will be appreciated that the disclosure also applies to computer programs, particularly computer programs on or in a carrier, adapted to put embodiments into practice. The program may be in the form of a source code, an object code, a code intermediate source and an object code such as in a partially compiled form, or in any other form suitable for use in the implementation of the method according to the embodiments described herein.

It will also be appreciated that such a program may have many different architectural designs. For example, a program code implementing the functionality of the method or system may be sub-divided into one or more sub-routines. Many different ways of distributing the functionality among these sub-routines will be apparent to the skilled person. The sub-routines may be stored together in one executable file to form a self-contained program. Such an executable file may include computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also include function calls to each other.

An embodiment relating to a computer program product includes computer-executable instructions corresponding to each processing stage of at least one of the methods set forth herein. These instructions may be sub-divided into sub-routines and/or stored in one or more files that may be linked statically or dynamically. Another embodiment relating to a computer program product includes computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer implemented method of training a model comprising:
receiving a first user input to annotate a first parameter in a portion of data;
using a first model to predict an annotation for at least one other parameter of the portion of data based on the received first user input for the first parameter; and
using the annotated first parameter, the predicted annotation of the at least one other parameter and the portion of data as training data to train a second model.

2. A method as in claim 1 wherein the second model is for annotating the first parameter and the at least one other parameter in a further portion of data.

3. A method as in claim 1 further comprising:
forming a training set of training data for training the second model by repeating:
receiving a first user input; and
using a first model to predict an annotation;
for a plurality of portions of data.

4. A method as in claim 1, wherein using a first model to predict an annotation is further based on the portion of data.

5. A method as in claim 1 further comprising:
receiving a second user input providing an indication of an accuracy of the predicted annotation of the at least one other parameter; and
using the indication of the accuracy of the predicted annotation as training data to train the second model.

6. A method as in claim 5 further comprising updating the first model based on the received second user input and the predicted annotation of the at least one other parameter.

7. A method as in claim 1 wherein using a first model to predict an annotation comprises:
using the first model to provide a plurality of suggestions for the annotation of the at least one other parameter; and
wherein the method further comprises:
receiving a third user input indicating an accuracy of at least one of the plurality of suggestions; and
using the indicated accuracy of the at least one of the plurality of suggestions as training data to train the second model.

8. A method as in claim 7 further comprising updating the first model based on the received third user input and the plurality of suggestions.

9. A method as in claim 1 wherein the predicted annotation of the at least one other parameter is based on confidence levels calculated by the first model.

10. A method as in claim 1 wherein:
the portion of data comprises an image;
the first parameter represents a location of a first feature in the image; and
the at least one other parameter represents locations of one or more other features in the image.

11. A method as in claim 1 wherein:
the portion of data comprises a sequence of images separated in time;
the first parameter relates to a first image in the sequence of images; and
the first model predicts an annotation of the first parameter and/or the at least one other parameter of the portion of data in a second image in the sequence of images, wherein the second image is a different image to the first image.

12. A method as in claim 1 wherein the portion of data comprises medical data.

13. A method as in claim 1 wherein the first and/or second model comprises a deep neural network.

14. A computer program product comprising a non-transitory computer readable medium, the computer readable medium having computer readable code embodied therein, the computer readable code being configured such that, on execution by a suitable computer or processor, the computer or processor is caused to perform the method of claim 1.

15. A system comprising:
a memory comprising instruction data representing a set of instructions;
a processor configured to communicate with the memory and to execute the set of instructions, wherein the set of instructions, when executed by the processor, cause the processor to:
receive a first user input to annotate a first parameter in a portion of data;
use a first model to predict an annotation for at least one other parameter of the portion of data based on the received first user input for the first parameter; and
use the annotated first parameter, the predicted annotation of the at least one other parameter and the portion of data as training data to train a second model.
